(19) United States
(12) Patent Application Publication
(10) Pub. No.: US 2011/0078826 A1
(43) Pub. Date: Mar. 31, 2011

(54) YIELD ENHANCEMENT IN PLANTS BY MODULATION OF A ZM-EREF-TS PROTEIN

(75) Inventors: Guofu Li, Johnston, IA (US); Jijun Zou, Johnston, IA (US)

(73) Assignee: PIONEER HI-BRED INTERNATIONAL, INC., Johnston, IA (US)

(21) Appl. No.: 12/891,860

(22) Filed: Sep. 28, 2010

Related U.S. Application Data
(60) Provisional application No. 61/247,025, filed on Sep. 30, 2009.

(51) Int. Cl. C12N 15/82; C07H 21/04; C07K 14/415; C12N 15/63; A01H 5/00

(52) U.S. Cl. 800/290; 536/23.6; 536/23.1; 530/370; 435/320.1; 800/320; 800/320.1; 800/320.2; 800/320.3; 800/278

(57) ABSTRACT

Compositions and methods for modulating plant development and for increasing yield in a plant are provided. The compositions include a Zm-EREF-Ts sequence. Compositions of the invention comprise amino acid sequences and nucleotide sequences selected from SEQ ID NOS: 1 and 2 as well as variants and fragments thereof. Nucleotide sequences encoding the Zm-EREF-Ts molecule are provided in DNA constructs for expression in a plant of interest are provided for modulating the level of a Zm-EREF-Ts sequence in a plant or a plant part are provided. The methods comprise introducing into a plant or plant part a heterologous polynucleotide comprising a Zm-EREF-Ts sequence of the invention. The level of the Zm-EREF-Ts polypeptide can be increased or decreased. Such method can be used to increase the yield in plants; in one embodiment, the method is used to increase grain yield in cereals.

### CROSS REFERENCE

This utility application claims the benefit U.S. Provisional Application No. 61/247,025, filed Sep. 30, 2009, which is incorporated herein by reference.

### FIELD OF THE INVENTION

The present invention is drawn to the field of genetics and molecular biology. More particularly, the compositions and methods are directed to modulation of transcription and improving yield in plants.

### BACKGROUND OF THE INVENTION

Grain yield improvements by conventional breeding have nearly reached a plateau in maize. It is natural then to explore some alternative, non-conventional approaches that could be employed to obtain further yield increases. Since the harvest index in maize has remained essentially unchanged during selection for grain yield over the last hundred or so years, the yield improvements have been realized from the increased total biomass production per unit land area (Sinclair, et al., (1998) Crop Science 38:638-643; Duvick, et al., (1999) Crop Science 39:1622-1630 and Tollenaar, et al., (1999) Crop Science 39:1597-1604). This increased total biomass has been achieved by increasing planting density, which has led to adaptive phenotypic alterations, such as a reduction in leaf angle and tassel size, the former to reduce shading of lower leaves and the latter perhaps to increase harvest index (Duvick, et al., (1999) Crop Science 39:1622-1630).

The Zm-EREF-Ts is a maize ethylene-responsive elongation factor. Constitutive over-expression of Zm-EREF-Ts has been shown to be able to enhance plant growth, increase kernel number and total kernel weight per plant. The overexpression of Zm-EREF-Ts would lead to increased grain yield in maize and other crop species. Zm-EREF-Ts contains a 28 amino acid region that is highly conserved and an elongation factor Ts signature.

Experimental data shows that the constitutively expressed Zm-EREF-Ts gene confers a strong positive effect on yield traits in maize, including enhanced kernel yield and increased grain yield.

Methods and compositions are needed in the art which can employ such sequences to modulate plant tissue growth and improve yield in plants.

### BRIEF SUMMARY OF THE INVENTION

Compositions and methods for modulating flower organ development, leaf formation, phototropism, apical dominance, fruit development, initiation of roots and for increasing yield in a plant are provided. The compositions include a Zm-EREF-Ts sequence. Compositions of the invention comprise amino acid sequences and nucleotide sequences selected from SEQ ID NOS: 1 and 2 as well as variants and fragments thereof.

Nucleotide sequences encoding the Zm-EREF-Ts are provided in DNA constructs for expression in a plant of interest. Expression cassettes, plants, plant cells, plant parts and seeds comprising the sequences of the invention are further provided. In specific embodiments, the polynucleotide is operably linked to a constitutive promoter.

Methods for modulating the level of a Zm-EREF-Ts sequence in a plant or a plant part are provided. The methods comprise introducing into a plant or plant part a heterologous polynucleotide comprising a Zm-EREF-Ts sequence or a Zm-EREF-Ts conserved region of the invention. The level of the Zm-EREF-Ts polypeptide can be increased or decreased. Such method can be used to increase the yield in plants; in one embodiment, the method is used to increase grain yield in cereals.

### BRIEF DESCRIPTION OF THE FIGURES

FIG. 1 provides an alignment of several Zm-EREF-Ts sequences from Zea mays (SEQ ID NO: 2), Arabidopsis thaliana (SEQ ID NO: 4), Oryza sativa (SEQ ID NO: 6), Populus trichocarpa (SEQ ID NO: 8) and Sorghum bicolor (SEQ ID NO: 10). The Zm-EREF-Ts consensus regions are indicated by highlighting. Bold font identifies amino acids with 100% identity; bold italics indicates amino acids with 75% identity and italics 50% identity between proteins. The 28 amino acid highly conserved region (SEQ ID NO: 12) and elongation factor Ts signature (SEQ ID NO: 13) are underlined with asterisks.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### I. Overview

Methods and compositions are provided to promote floral organ development, root initiation and yield and for modulating leaf formation, phototropism, apical dominance, fruit development and the like, in plants. The compositions and methods of the invention result in improved plant or crop yield by modulating in a plant the level of at least one Zm-EREF-Ts polypeptide or a polypeptide having a biologically active variant or fragment of a Zm-EREF-Ts polypeptide of the invention.

### II. Compositions

Compositions of the invention include Zm-EREF-Ts polynucleotides and polypeptides and variants and fragments thereof that are involved in regulating transcription.

TABLE 1

| | SEQUENCE ID |
|---|---|
| SEQUENCE NAME | NUMBER |
| Zea may ZM-EREF-TS polynucleotide | SEQ ID NO: 1 |
| Zea may ZM-EREF-TS polypeptide | SEQ ID NO: 2 |
| Arabidopsis thaliana At4g11120.1 polynucleotide | SEQ ID NO: 3 |
| Arabidopsis thaliana At4g11120.1 polypeptide | SEQ ID NO: 4 |
| Oryza sativa polynucleotide | SEQ ID NO: 5 |
| Oryza sativa polypeptide | SEQ ID NO: 6 |
| Populus trichocarpa Pt_807701 polynucleotide | SEQ ID NO: 7 |
| Populus trichocarpa Pt_807701 polypeptide | SEQ ID NO: 8 |
| Sorghum bicolor Sb03g141510 polynucleotide | SEQ ID NO: 9 |
| Sorghum bicolor Sb03g141510 polypeptide | SEQ ID NO: 10 |
| Zea mays ZM-EREF-TS CDS sequence | SEQ ID NO: 11 |
| Highly conserved polypeptide sequence | SEQ ID NO: 12 |
| Elongation factor Ts signature polypeptide | SEQ ID NO: 13 |

By “corresponding to” is intended that the recited amino acid positions for each domain relate to the amino acid positions of the recited SEQ ID NO and that polypeptides comprising these domains may be found by aligning the polypeptides with the recited SEQ ID NO using standard alignment methods.

The Zm-EREF-Ts sequences of the invention have been identified as plant growth and development related sequences. Zm-EREF-Ts is expressed in all tissues tested throughout plant development.

As used herein, a “Zm-EREF-Ts” or “Zm-EREF-Ts” sequence comprises a polynucleotide encoding a Zm-EREF-Ts polypeptide or a polypeptide having the Zm-EREF-Ts conserved region or a biologically active variant or fragment of the Zm-EREF-Ts or Zm-EREF-Ts conserved region.

In one embodiment, the present invention provides isolated Zm-EREF-Ts polypeptides comprising amino acid sequences as shown in SEQ ID NO: 2 and fragments and variants thereof. Further provided are polynucleotides comprising the nucleotide sequence set forth in SEQ ID NO: 1.

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized.

Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

Fragments and variants of the Zm-EREF-Ts conserved region or Zm-EREF-Ts polynucleotides and proteins encoded thereby are also encompassed by the methods and compositions of the present invention. By “fragment” is intended a portion of the polynucleotide or a portion of the amino acid sequence. Fragments of a polynucleotide may encode protein fragments that retain the biological activity of the native protein and hence regulate transcription. For example, polypeptide fragments will comprise the Zm-EREF-Ts conserved region. Alternatively, fragments that are used for suppressing or silencing (i.e., decreasing the level of expression) of a Zm-EREF-Ts sequence need not encode a protein fragment, but will retain the ability to suppress expression of the target sequence. In addition, fragments that are useful as hybridization probes generally do not encode fragment proteins retaining biological activity. Thus, fragments of a nucleotide sequence may range from at least about 18 nucleotides, about 20 nucleotides, about 50 nucleotides, about 100 nucleotides and up to the full-length polynucleotide encoding the proteins of the invention.

A fragment of a polynucleotide encoding a Zm-EREF-Ts conserved region or a Zm-EREF-Ts polypeptide will encode at least 15, 25, 30, 50, 100, 150, 200, 250, 300, 350 contiguous amino acids or up to the total number of amino acids present in a full-length Zm-EREF-Ts conserved region or Zm-EREF-Ts protein (i.e., SEQ ID NO: 2, 379 amino acids). Fragments of a Zm-EREF-Ts conserved region or a Zm-EREF-Ts polynucleotide that are useful as hybridization probes, PCR primers or as suppression constructs generally need not encode a biologically active portion of a Zm-EREF-Ts protein or a Zm-EREF-Ts conserved region.

A biologically active portion of a polypeptide comprising a Zm-EREF-Ts conserved region, or a Zm-EREF-Ts protein can be prepared by isolating a portion of a Zm-EREF-Ts polynucleotide, expressing the encoded portion of the Zm-EREF-Ts protein (e.g., by recombinant expression in vitro) and assessing the activity of the encoded portion of the Zm-EREF-Ts protein. Polynucleotides that are fragments of a Zm-EREF-Ts nucleotide sequence, or a polynucleotide sequence comprising a Zm-EREF-Ts conserved region comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500 contiguous nucleotides or up to the number of nucleotides present in a Zm-EREF-Ts conserved region or in a Zm-EREF-Ts polynucleotide (i.e., SEQ ID NO: 1, 1524 nucleotides).

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the Zm-EREF-Ts polypeptides or a Zm-EREF-Ts conserved region. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotide, such as those generated, for example, by using site-directed mutagenesis but which still encode a polypeptide comprising a Zm-EREF-Ts conserved region or a Zm-EREF-Ts polypeptide that is capable of regulating transcription or that is capable of reducing the level of expression (i.e., suppressing or silencing) of a Zm-EREF-Ts polynucleotide. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO. 1 or SEQ ID NO: 2 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, that is, regulate transcription as described herein. Such variants may result from, for example, genetic polymorphism or from human manipulation. Biologically active variants of a Zm-EREF-Ts protein of the invention or a Zm-EREF-Ts conserved region will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the Zm-EREF-Ts protein or the Zm-EREF-Ts conserved region as determined by sequence alignment programs and parameters described elsewhere herein. A biologically active variant of a Zm-EREF-Ts protein of the invention or of a Zm-EREF-Ts conserved region may differ from that protein by as few as 1-15 amino acid residues, as few as 1-10, such as 6-10, as few as 5, as few as 4, 3, 2 or even 1 amino acid residue.

The polynucleotides of the invention may be altered in various ways including amino acid substitutions, deletions, truncations and insertions. Methods for such manipulations are generally known in the art. For example, amino acid sequence variants and fragments of the Zm-EREF-Ts proteins or Zm-EREF-Ts conserved regions can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found, Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired activity (i.e., the ability to regulate transcription or decrease the level of expression of a target Zm-EREF-Ts sequence). In specific embodiments, the mutations that will be made in the DNA encoding the variant do not place the sequence out of reading frame and do not create complementary regions that could produce secondary mRNA structure. See, EP Patent Application Publication Number 75,444.

The deletions, insertions and substitutions of the protein sequences encompassed herein are not expected to produce radical changes in the characteristics of the protein. However, when it is difficult to predict the exact effect of the substitution, deletion or insertion in advance of doing so, one skilled in the art will appreciate that the effect will be evaluated by routine screening assays. For example, the activity of a Zm-EREF-Ts polypeptide can be evaluated by assaying for the ability of the polypeptide to regulate transcription. Various methods can be used to assay for this activity, including, directly monitoring the level of expression of a target gene at the nucleotide or polypeptide level. Methods for such an analysis are known and include, for example, Northern blots, 51 protection assays, Western blots, enzymatic or colorimetric assays. Alternatively, methods to assay for a modulation of transcriptional activity can include monitoring for an alteration in the phenotype of the plant. For example, as discussed in further detail elsewhere herein, modulating the level of a Zm-EREF-Ts polypeptide can result in modulation of flower formation, root initiation and alteration of yield. Methods to assay for these changes are discussed in further detail elsewhere herein.

Variant polynucleotides and proteins also encompass sequences and proteins derived from a mutagenic and recombinogenic procedure such as DNA shuffling. With such a procedure, one or more different Zm-EREF-Ts coding sequences can be manipulated to create a new Zm-EREF-Ts sequence or Zm-EREF-Ts conserved region possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the Zm-EREF-Ts gene of the invention and other known Zm-EREF-Ts genes to obtain a new gene coding for a protein with an improved property of interest, such as an increased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458.

The polynucleotides of the invention can be used to isolate corresponding sequences from other organisms, particularly other plants, more particularly other monocots. In this manner, methods such as PCR, hybridization and the like can be used to identify such sequences based on their sequence homology to the sequences set forth herein. Sequences isolated based on their sequence identity to the entire Zm-EREF-Ts sequences or Zm-EREF-Ts conserved regions of the present invention set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99% or greater sequence identity. Functions of orthologs are often highly conserved among species. Thus, isolated polynucleotides that can silence or suppress the expression of a Zm-EREF-Ts sequence or a polynucleotide that encodes for a Zm-EREF-Ts protein and which hybridize under stringent conditions to the Zm-EREF-Ts sequences disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook, et al., (1989) Molecular Cloning: A Laboratory Manual (2d ed., Cold Spring Harbor Laboratory Press, Plainview, N.Y.). See also, Innis, et al., eds. (1990) PCR Protocols: A Guide to Methods and Applications (Academic Press, New York); Innis and Gelfand, eds. (1995) PCR Strategies (Academic Press, New York) and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments or other oligonucleotides and may be labeled with a detectable group such as ³²P or any other detectable marker. Thus, for example, probes for hybridization can be made by labeling synthetic oligonucleotides based on the Zm-EREF-Ts polynucleotides of the invention. Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, N.Y.).

For example, the entire Zm-EREF-Ts polynucleotide or a polynucleotide encoding a Zm-EREF-Ts conserved region disclosed herein, or one or more portions thereof, may be used as a probe capable of specifically hybridizing to corresponding Zm-EREF-Ts polynucleotide and messenger RNAs. To achieve specific hybridization under a variety of conditions, such probes include sequences that are unique among Zm-EREF-Ts polynucleotide sequences and are optimally at least about 10 nucleotides in length and most optimally at least about 20 nucleotides in length. Such probes may be used to amplify corresponding Zm-EREF-Ts polynucleotide from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook, et al., (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching, thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, N.Y.) and Ausubel, et al., eds. (1995) Current Protocols in Molecular Biology, Chapter 2 (Greene Publishing and Wiley-Interscience, New York). See, Sambrook, et al., (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package®, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12 and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2 and the BLOSUM62 scoring matrix or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453 to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package® is BLOSUM62 (see, Henikoff and Henikoff, (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

B. Plants

In specific embodiments, the invention provides plants, plant cells and plant parts having altered levels (i.e., an increase or decrease) of a Zm-EREF-Ts sequence. In some embodiments, the plants and plant parts have stably incorporated into their genome at least one heterologous polynucleotide encoding a Zm-EREF-Ts polypeptide comprising the Zm-EREF-Ts conserved region as set forth in SEQ ID NO: 3 or a biologically active variant or fragment thereof. In one embodiment, the polynucleotide encoding the Zm-EREF-Ts polypeptide is set forth in SEQ ID NO: 1 or a biologically active variant or fragment thereof.

In yet other embodiments, plants and plant parts are provided in which the heterologous polynucleotide stably integrated into the genome of the plant or plant part comprises a polynucleotide which when expressed in a plant increases the level of a Zm-EREF-Ts polypeptide comprising a Zm-EREF-Ts conserved region or an active variant or fragment thereof. Sequences that can be used to increase expression of a Zm-EREF-Ts polypeptide include, but are not limited to, the sequence set forth in SEQ ID NO: 1 or variants or fragments thereof.

As discussed in further detail elsewhere herein, such plants, plant cells, plant parts and seeds can have an altered phenotype including, for example, altered flower organ development, leaf formation, phototropism, apical dominance, fruit development, root initiation and improved yield.

As used herein, the term plant includes plant cells, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, kernels, ears, cobs, husks, stalks, roots, root tips, anthers and the like. Grain is intended to mean the mature seed produced by commercial growers for purposes other than growing or reproducing the species. Progeny, variants and mutants of the regenerated plants are also included within the scope of the invention, provided that these parts comprise the introduced or heterologous polynucleotides disclosed herein.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.) and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus elliotil), ponderosa pine (Pinus ponderosa), lodgepole pine (Pinus contorta) and Monterey pine (Pinus radiata), Douglas-fir (Pseudotsuga menziesii), Western hemlock (Tsuga canadensis), Sitka spruce (Picea glauca), redwood (Sequoia sempervirens), true firs such as silver fir (Abies amabilis) and balsam fir (Abies balsamea) and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “subject plant or plant cell” is one in which an alteration, such as transformation or introduction of a polypeptide, has occurred, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed.

C. Polynucleotide Constructs

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures and the like.

The various polynucleotides employed in the methods and compositions of the invention can be provided in expression cassettes for expression in the plant of interest. The cassette will include 5′ and 3′ regulatory sequences operably linked to a polynucleotide of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the Zm-EREF-Ts polynucleotide to be under the transcriptional regulation of the regulatory regions. The expression cassette may additionally contain selectable marker genes.

The expression cassette can include in the 5′-3′ direction of transcription, a transcriptional and translational initiation region (i.e., a promoter), a Zm-EREF-Ts polynucleotide and a transcriptional and translational termination region (i.e., termination region) functional in plants. The regulatory regions (i.e., promoters, transcriptional regulatory regions and translational termination regions) and/or the Zm-EREF-Ts polynucleotide may be native/analogous to the host cell or to each other. Alternatively, the regulatory regions and/or the Zm-EREF-Ts polynucleotides may be heterologous to the host cell or to each other. As used herein, “heterologous” in reference to a sequence is a sequence that originates from a foreign species or, if from the same species, is substantially modified from its native form in composition and/or genomic locus by deliberate human intervention. For example, a promoter operably linked to a heterologous polynucleotide is from a species different from the species from which the polynucleotide was derived or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While it may be optimal to express the sequences using heterologous promoters, the native promoter sequences may be used. Such constructs can change expression levels of a Zm-EREF-Ts transcript or protein in the plant or plant cell. Thus, the phenotype of the plant or plant cell can be altered.

The termination region may be native with the transcriptional initiation region, may be native with the operably linked Zm-EREF-Ts polynucleotide of interest, may be native with the plant host or may be derived from another source (i.e., foreign or heterologous) to the promoter, the Zm-EREF-Ts polynucleotide of interest, the plant host or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20), and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

A number of promoters can be used in the practice of the invention, including the native promoter of the polynucleotide sequence of interest. The promoters can be selected based on the desired outcome. The nucleic acids can be combined with constitutive, tissue-preferred or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026), GOS2 promoter (dePater, et al., (1992) Plant J. 2:837-44) and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional selectable markers include phenotypic markers such as 13-galactosidase and fluorescent proteins such as green fluorescent protein (GFP) (Su, et al., (2004) Biotechnol Bioeng 85:610-9 and Fetter, et al., (2004) Plant Cell 16.215-28), cyan florescent protein (CYP) (Bolte, et al., (2004) J. Cell Science 117:943-54 and Kato, et al., (2002) Plant Physiol 129:913-42) and yellow florescent protein (PhiYFP™ from Evrogen, see, Bolte, et al., (2004) J. Cell Science 117:943-54). For additional selectable markers, see generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the present invention.

In certain embodiments the polynucleotides of the present invention can be stacked with any combination of polynucleotide sequences of interest in order to create plants with a desired trait. A trait, as used herein, refers to the phenotype derived from a particular sequence or groups of sequences. The combinations generated can also include multiple copies of any one of the polynucleotides of interest. The polynucleotides of the present invention can also be stacked with traits desirable for disease or herbicide resistance (e.g., fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE), and starch debranching enzymes (SDBE)) and polymers or bioplastics (e.g., U.S. Pat. No. 5,602,321; beta-ketothiolase, polyhydroxybutyrate synthase and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides providing agronomic traits such as male sterility (e.g., see U.S. Pat. No. 5,583,210), stalk strength, flowering time or transformation technology traits such as cell cycle regulation or gene targeting (e.g., WO 99/61619, WO 00/17364 and WO 99/25821), the disclosures of which are herein incorporated by reference.

These stacked combinations can be created by any method including, but not limited to, cross-breeding plants by any conventional or TopCross methodology or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855 and WO99/25853, all of which are herein incorporated by reference.

D. Method of Introducing

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods and virus-mediated methods.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722) and ballistic particle acceleration (see, for example, U.S. Pat. No. 4,945,050; U.S. Pat. No. 5,879,918; U.S. Pat. Nos. 5,886,244 and 5,932,782; Tomes, et al., (1995) in Plant Cell, Tissue, and Organ Culture: Fundamental Methods, ed. Gamborg and Phillips, (Springer-Verlag, Berlin); McCabe, et al., (1988) Biotechnology 6:923-926) and Lec1 transformation (WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference.

In specific embodiments, the Zm-EREF-Ts sequences or variants and fragments thereof can be provided to a plant using a variety of transient transformation methods. Such transient transformation methods include, but are not limited to, the introduction of the Zm-EREF-Ts protein or variants and fragments thereof directly into the plant or the introduction of the Zm-EREF-Ts transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, the Zm-EREF-Ts polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In other embodiments, the polynucleotide of the invention may be introduced into plants by contacting plants with a virus or viral nucleic acids. Generally, such methods involve incorporating a nucleotide construct of the invention within a viral DNA or RNA molecule. It is recognized that the a Zm-EREF-Ts sequence or a variant or fragment thereof may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931 and Porta, et al., (1996) Molecular Biotechnology 5:209-221, herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855 and WO99/25853, all of which are herein incorporated by reference. Briefly, the polynucleotide of the invention can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into their genome.

### III. Methods of Use

A. Methods for Modulating Expression of at Least One Zm-EREF-Ts Sequence or a Variant or Fragment Therefore in a Plant or Plant Part

A “modulated level” or “modulating level” of a polypeptide in the context of the methods of the present invention refers to any increase or decrease in the expression, concentration or activity of a gene product, including any relative increment in expression, concentration or activity. Any method or composition that modulates expression of a target gene product, either at the level of transcription or translation or modulates the activity of the target gene product can be used to achieve modulated expression, concentration, activity of the target gene product. In general, the level is increased or decreased by at least 1%, 5%, 10%, 20%, 30%, 40%, 50%, 60%, 70%, 80%, 90% or greater relative to an appropriate control plant, plant part or cell. Modulation in the present invention may occur during and/or subsequent to growth of the plant to the desired stage of development. In specific embodiments, the polypeptides of the present invention are modulated in monocots, particularly grain plants such as rice, wheat, maize and the like.

The expression level of a polypeptide having a Zm-EREF-Ts conserved region or a biologically active variant or fragment thereof may be measured directly, for example, by assaying for the level of the Zm-EREF-Ts polypeptide in the plant or indirectly, for example, by measuring the level of the polynucleotide encoding the protein or by measuring the activity of the Zm-EREF-Ts polypeptide in the plant. Methods for determining the activity of the Zm-EREF-Ts polypeptide are described elsewhere herein.

In specific embodiments, the polypeptide or the polynucleotide of the invention is introduced into the plant cell. Subsequently, a plant cell having the introduced sequence of the invention is selected using methods known to those of skill in the art such as, but not limited to, Southern blot analysis, DNA sequencing, PCR analysis or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,984, all of which are herein incorporated by reference. See also, WO 98/49350, WO 99/07865, WO 99/25821 and Beetham, et al. (1999) Proc. Natl. Acad. Sci. USA 96:8774-8778, herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions and substitutions of nucleotides into the genome. While the methods of the present invention do not depend on additions, deletions and substitutions of any particular number of nucleotides, it is recognized that such additions, deletions or substitutions comprises at least one nucleotide.

In one embodiment, the activity and/or level of a Zm-EREF-Ts polypeptide is increased. An increase in the level and/or activity of the Zm-EREF-Ts polypeptide can be achieved by providing to the plant a Zm-EREF-Ts polypeptide or a biologically active variant or fragment thereof. As discussed elsewhere herein, many methods are known in the art for providing a polypeptide to a plant including, but not limited to, direct introduction of the Zm-EREF-Ts polypeptide into the plant or introducing into the plant (transiently or stably) a polynucleotide construct encoding a polypeptide having Zm-EREF-Ts activity. It is also recognized that the methods of the invention may employ a polynucleotide that is not capable of directing in the transformed plant the expression of a protein or an RNA. Thus, the level and/or activity of a Zm-EREF-Ts polypeptide may be increased by altering the gene encoding the Zm-EREF-Ts polypeptide or its promoter. See, e.g., Kmiec, U.S. Pat. No. 5,565,350; Zarling, et al., PCT/US93/03868. Therefore, mutagenized plants that carry mutations in Zm-EREF-Ts genes, where the mutations increase expression of the Zm-EREF-Ts gene or increase the activity of the encoded Zm-EREF-Ts polypeptide, are provided.

In other embodiments, the activity and/or level of the Zm-EREF-Ts polypeptide of the invention is reduced or eliminated by introducing into a plant a polynucleotide that inhibits the level or activity of a polypeptide. The polynucleotide may inhibit the expression of Zm-EREF-Ts gene directly, by preventing translation of the Zm-EREF-Ts messenger RNA, or indirectly, by encoding a polypeptide that inhibits the transcription or translation of a Zm-EREF-Ts gene encoding a Zm-EREF-Ts protein. Methods for inhibiting or eliminating the expression of a gene in a plant are well known in the art and any such method may be used in the present invention to inhibit the expression of at least one Zm-EREF-Ts sequence in a plant. In other embodiments of the invention, the activity of a Zm-EREF-Ts polypeptide is reduced or eliminated by transforming a plant cell with a sequence encoding a polypeptide that inhibits the activity of the Zm-EREF-Ts polypeptide. In other embodiments, the activity of a Zm-EREF-Ts polypeptide may be reduced or eliminated by disrupting the gene encoding the Zm-EREF-Ts polypeptide. The invention encompasses mutagenized plants that carry mutations in Zm-EREF-Ts genes, where the mutations reduce expression of the Zm-EREF-Ts gene or inhibit the Zm-EREF-Ts activity of the encoded Zm-EREF-Ts polypeptide.

Reduction of the activity of specific genes (also known as gene silencing or gene suppression) is desirable for several aspects of genetic engineering in plants. Many techniques for gene silencing are well known to one of skill in the art, including, but not limited to, antisense technology (see, e.g., Sheehy, et al., (1988) Proc. Natl. Acad. Sci. USA 85:8805-8809 and U.S. Pat. Nos. 5,107,065; 5,453,566 and 5,759,829); cosuppression (e.g., Taylor, (1997) Plant Cell 9:1245; Jorgensen, (1990) Trends Biotech. 8(12):340-344; Flavell, (1994) Proc. Natl. Acad. Sci. USA 91:3490-3496; Finnegan, et al., (1994) Bio/Technology 12:883-888 and Neuhuber, et al., (1994) Mol. Gen. Genet. 244:230-241); RNA interference (Napoli, et al., (1990) Plant Cell 2:279-289; U.S. Pat. No. 5,034,323; Sharp, (1999) Genes Dev. 13:139-141; Zamore, et al., (2000) Cell 101:25-33 and Montgomery, et al., (1998) Proc. Natl. Acad. Sci. USA 95:15502-15507), virus-induced gene silencing (Burton, et al., (2000) Plant Cell 12:691-705 and Baulcombe, (1999) Curr. Op. Plant Bio. 2:109-113); target-RNA-specific ribozymes (Haseloff, et al., (1988) Nature 334:585-591); hairpin structures (Smith, et al., (2000) Nature 407:319-320; WO 99/53050; WO 02/00904; WO 98/53083; Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7, US Patent Application Publication Number 2003/0175965; Panstruga, et al., (2003) Mol. Biol. Rep. 30:135-140; Wesley, et al., (2001) Plant J. 27:581-590; Wang and Waterhouse, (2001) Curr. Opin. Plant Biol. 5:146-150; US Patent Application Publication Number 2003/0180945 and WO 02/00904, all of which are herein incorporated by reference); ribozymes (Steinecke, et al., (1992) EMBO J. 11:1525 and Perriman, et al., (1993) Antisense Res. Dev. 3:253); oligonucleotide-mediated targeted modification (e.g., WO 03/076574 and WO 99/25853); Zn-finger targeted molecules (e.g., WO 01/52620; WO 03/048345 and WO 00/42219); transposon tagging (Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928; Bensen, et al., (1995) Plant Cell 7:75-84; Mena, et al., (1996) Science 274:1537-1540 and U.S. Pat. No. 5,962,764), each of which is herein incorporated by reference and other methods or combinations of the above methods known to those of skill in the art.

It is recognized that with the polynucleotides of the invention, antisense constructions, complementary to at least a portion of the messenger RNA (mRNA) for the Zm-EREF-Ts sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used.

The polynucleotides of the present invention may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

Thus, many methods may be used to reduce or eliminate the activity of a Zm-EREF-Ts polypeptide or a biologically active variant or fragment thereof. In addition, combinations of methods may be employed to reduce or eliminate the activity of at least one Zm-EREF-Ts polypeptide. It is further recognized that the level of a single Zm-EREF-Ts sequence can be modulated to produce the desired phenotype. Alternatively, is may be desirable to modulate (increase and/or decrease) the level of expression of a biologically active variant or fragment thereof.

As discussed above, a variety of promoters can be employed to modulate the level of the Zm-EREF-Ts sequence. In one embodiment, the expression of the heterologous polynucleotide which modulates the level of at least one Zm-EREF-Ts polypeptide can be regulated by a tissue-preferred promoter, particularly, a leaf-preferred promoter (i.e., mesophyll-preferred promoter or a bundle sheath preferred promoter) and/or a seed-preferred promoter (i.e., an endosperm-preferred promoter or an embryo-preferred promoter).

B. Methods to Modulate Floral Organ Development and Yield in a Plant

Methods and compositions are provided to modulate Zm-EREF-Ts and Zm-EREF-Ts polypeptides and thus to modulate floral organ development, root initiation and yield in plants. In one embodiment, the compositions of the invention can be used to increase grain yield in cereal plants. In this embodiment, the Zm-EREF-Ts coding sequence is expressed in a cereal plant of interest to increase expression of the Zm-EREF-Ts transcription factor.

In this manner, the methods and compositions can be used to increase yield in a plant. As used herein, the term “improved yield” means any improvement in the yield of any measured plant product. The improvement in yield can comprise a 0.1%, 0.5%, 1%, 3%, 5%, 10%, 15%, 20%, 30%, 40%, 50%, 60%, 70%, 80%, 90% or greater increase in measured plant product. Alternatively, the increased plant yield can comprise about a 0.5 fold, 1 fold, 2 fold, 4 fold, 8 fold, 16 fold or 32 fold increase in measured plant products. For example, an increase in the bu/acre yield of soybeans or corn derived from a crop having the present treatment as compared with the bu/acre yield from untreated soybeans or corn cultivated under the same conditions would be considered an improved yield. By increased yield is also intended at least one of an increase in total seed numbers, an increase in total seed weight, an increase in root biomass and an increase in harvest index. Harvest index is defined as the ratio of yield biomass to the total cumulative biomass at harvest.

Accordingly, various methods to increase yield of a plant are provided. In one embodiment, increasing yield of a plant or plant part comprises introducing into the plant or plant part a heterologous polynucleotide and expressing the heterologous polynucleotide in the plant or plant part. In this method, the expression of the heterologous polynucleotide modulates the level of at least one Zm-EREF-Ts polypeptide in the plant or plant part.

In specific embodiments, modulation of the level of the Zm-EREF-Ts polypeptide comprises an increase in the level of at least one Zm-EREF-Ts polypeptide. In such methods, the heterologous polynucleotide introduced into the plant encodes a polypeptide having a Zm-EREF-Ts conserved region or a biologically active variant or fragment thereof. In specific embodiments, the heterologous polynucleotide comprises the sequence set forth in at least one SEQ ID NO: 1 and/or a biologically active variant or fragment thereof.

In other embodiments, modulating the level of at least one Zm-EREF-Ts polypeptide comprises decreasing in the level of at least one Zm-EREF-Ts polypeptide. In such methods, the heterologous polynucleotide introduced into the plant need not encode a functional Zm-EREF-Ts polypeptide, but rather the expression of the polynucleotide results in the decreased expression of a Zm-EREF-Ts polypeptide comprising a biologically active variant or fragment of the Zm-EREF-Ts conserved region. In specific embodiments, the Zm-EREF-Ts polypeptide having the decreased level is set forth in at least one of SEQ ID NO: 2 or a biologically active variant or fragment thereof.

The following examples are offered by way of illustration and not by way of limitation.

### EXPERIMENTAL

### Example 1

### Cloning of Maize Zm-EREF-Ts Gene

The cDNA that encoded the Zm-EREF-Ts polypeptide from maize was identified by sequence homology from a collection of ESTs generated from a maize cDNA library using BLAST 2.0 (Altschul, et al., (1990) J. Mol. Biol. 215:403) against the NCBI DNA sequence database. From the EST plasmid, the maize Zm-EREF-Ts cDNA fragment nucleotide #394 to #1533 of SEQ ID NO: 1 was amplified by PCR using Hifi Taq DNA polymerase in standard conditions with maize Zm-EREF-Ts-specific primers that included the AttB site for GATEWAY® recombination cloning. A PCR fragment of the expected length was amplified and purified using standard methods as described by Sambrook, et al., (1989) Molecular Cloning: A Laboratory Manual (2d ed., Cold Spring Harbor Laboratory Press, Plainview, N.Y.). The first step of the GATEWAY® procedure, the BP reaction, was then performed, during which the PCR fragment recombined in vivo with the pDONR201 plasmid to produce the “entry clone.” Plasmid pDONR201 was purchased from Invitrogen, as part of the GATEWAY® technology (Invitrogen, Carlsbad, Calif.).

### Example 2

### Overexpression of Zm-EREF-Ts Sequences in Maize

Immature maize embryos from greenhouse donor plants are transformed with a plasmid containing a Zm-EREF-Ts sequence (such as Zm-EREF-Ts/SEQ ID NO: 1) under the control of the UBI promoter and the selectable marker gene PAT (Wohlleben, et al., (1988) Gene 70:25-37), which confers resistance to the herbicide Bialaphos. Alternatively, the selectable marker gene is provided on a separate plasmid. Transformation is performed as follows. Media recipes follow below.

### Preparation of Target Tissue

The ears are husked and surface sterilized in 30% Clorox® bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector comprising the Zm-EREF-Ts sequence operably linked to a ubiquitin promoter is made. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaC1₂ and 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol and centrifuged for 30 seconds. Again the liquid is removed and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun (U.S. Pat. No. 5,240,855). All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for an increase in nitrogen use efficiency, increase yield, or an increase in stress tolerance.

Bombardment medium (560Y) comprises 4.0 g/l N6 basal salts (SIGMA C-1416), 1.0 ml/l Eriksson's Vitamin Mix (1000×SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite® (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000×SIGMA-1511), 0.5 mg/l thiamine HCl, 30.0 g/l sucrose and 2.0 mg/l 2,4-D (brought to volume with D-I H₂O following adjustment to pH 5.8 with KOH); 3.0 g/l Gelrite® (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose and 1.0 ml/l of 0.1 mM abscisic acid (brought to volume with polished D-I H₂O after adjusting to pH 5.6); 3.0 g/l Gelrite® (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL and 0.40 g/l glycine brought to volume with polished D-I H₂O), 0.1 g/1 myo-inositol and 40.0 g/l sucrose (brought to volume with polished D-I H₂O after adjusting pH to 5.6) and 6 g/l Bacto™-agar (added after bringing to volume with polished D-I H₂O), sterilized and cooled to 60° C.

### Example 3

### Agrobacterium-Mediated Transformation

For Agrobacterium-mediated transformation of maize with a Zm-EREF-Ts polynucleotide the method of Zhao is employed (U.S. Pat. No. 5,981,840 and PCT patent publication WO98/32326, the contents of which are hereby incorporated by reference). Briefly, immature embryos are isolated from maize and the embryos contacted with a suspension of Agrobacterium, where the bacteria are capable of transferring the Zm-EREF-Ts polynucleotide to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step) and calli grown on selective medium are cultured on solid medium to regenerate the plants.

### Example 4

### Soybean Embryo Transformation

### Culture Conditions

Soybean embryogenic suspension cultures (cv. Jack) are maintained in 35 ml liquid medium SB196 (see, recipes below) on rotary shaker, 150 rpm, 26° C. with cool white fluorescent lights on 16:8 hr day/night photoperiod at light intensity of 60-85 μE/m2/s. Cultures are subcultured every 7 days to two weeks by inoculating approximately 35 mg of tissue into 35 ml of fresh liquid SB196 (the preferred subculture interval is every 7 days).

Soybean embryogenic suspension cultures are transformed with the plasmids and DNA fragments described in the following examples by the method of particle gun bombardment (Klein, et al., (1987) Nature, 327:70).

### Soybean Embryogenic Suspension Culture Initiation

Soybean cultures are initiated twice each month with 5-7 days between each initiation.

Pods with immature seeds from available soybean plants 45-55 days after planting are picked, removed from their shells and placed into a sterilized magenta box. The soybean seeds are sterilized by shaking them for 15 minutes in a 5% Clorox® solution with 1 drop of ivory soap (95 ml of autoclaved distilled water plus 5 ml Clorox® and 1 drop of soap). Mix well. Seeds are rinsed using 2 1-liter bottles of sterile distilled water and those less than 4 mm are placed on individual microscope slides. The small end of the seed are cut and the cotyledons pressed out of the seed coat. Cotyledons are transferred to plates containing SB1 medium (25-30 cotyledons per plate). Plates are wrapped with fiber tape and stored for 8 weeks. After this time secondary embryos are cut and placed into SB196 liquid media for 7 days.

### Preparation of DNA for Bombardment

Either an intact plasmid or a DNA plasmid fragment containing the genes of interest and the selectable marker gene are used for bombardment. Plasmid DNA for bombardment are routinely prepared and purified using the method described in the Promega™ Protocols and Applications Guide, Second Edition (page 106). Fragments of the plasmids carrying a Zm-EREF-Ts polynucleotide are obtained by gel isolation of double digested plasmids. In each case, 100 μg of plasmid DNA is digested in 0.5 ml of the specific enzyme mix that is appropriate for the plasmid of interest. The resulting DNA fragments are separated by gel electrophoresis on 1% SeaPlaque GTG agarose (BioWhitaker Molecular Applications) and the DNA fragments containing the Zm-EREF-Ts polynucleotide are cut from the agarose gel. DNA is purified from the agarose using the GELase digesting enzyme following the manufacturer's protocol.

A 50 μl aliquot of sterile distilled water containing 3 mg of gold particles (3 mg gold) is added to 5 μl of a 1 μg/μl DNA solution (either intact plasmid or DNA fragment prepared as described above), 50 μl 2.5M CaCl₂ and 20 μl of 0.1 M spermidine. The mixture is shaken 3 min on level 3 of a vortex shaker and spun for 10 sec in a bench microfuge. After a wash with 400 μl 100% ethanol the pellet is suspended by sonication in 40 μl of 100% ethanol. Five μl of DNA suspension is dispensed to each flying disk of the Biolistic PDS1000/HE instrument disk. Each 5 μl aliquot contains approximately 0.375 mg gold per bombardment (i.e., per disk).

Tissue Preparation and Bombardment with DNA

Approximately 150-200 mg of 7 day old embryonic suspension cultures are placed in an empty, sterile 60×15 mm petri dish and the dish covered with plastic mesh. Tissue is bombarded 1 or 2 shots per plate with membrane rupture pressure set at 1100 PSI and the chamber evacuated to a vacuum of 27-28 inches of mercury. Tissue is placed approximately 3.5 inches from the retaining/stopping screen.

### Selection of Transformed Embryos

Transformed embryos were selected either using hygromycin (when the hygromycin phosphotransferase, HPT, gene was used as the selectable marker) or chlorsulfuron (when the acetolactate synthase, ALS, gene was used as the selectable marker).

### Hygromycin (HPT) Selection

Following bombardment, the tissue is placed into fresh SB196 media and cultured as described above. Six days post-bombardment, the SB196 is exchanged with fresh SB196 containing a selection agent of 30 mg/L hygromycin. The selection media is refreshed weekly. Four to six weeks post selection, green, transformed tissue may be observed growing from untransformed, necrotic embryogenic clusters. Isolated, green tissue is removed and inoculated into multiwell plates to generate new, clonally propagated, transformed embryogenic suspension cultures.

### Chlorsulfuron (ALS) Selection

Following bombardment, the tissue is divided between 2 flasks with fresh SB196 media and cultured as described above. Six to seven days post-bombardment, the SB196 is exchanged with fresh SB196 containing selection agent of 100 ng/ml Chlorsulfuron. The selection media is refreshed weekly. Four to six weeks post selection, green, transformed tissue may be observed growing from untransformed, necrotic embryogenic clusters. Isolated, green tissue is removed and inoculated into multiwell plates containing SB196 to generate new, clonally propagated, transformed embryogenic suspension cultures.

Regeneration of Soybean Somatic Embryos into Plants

In order to obtain whole plants from embryogenic suspension cultures, the tissue must be regenerated.

### Embryo Maturation

Embryos are cultured for 4-6 weeks at 26° C. in SB196 under cool white fluorescent (Phillips cool white Econowatt F40/CW/RS/EW) and Agro (Phillips F40 Agro) bulbs (40 watt) on a 16:8 hr photoperiod with light intensity of 90-120 uE/m2s. After this time embryo clusters are removed to a solid agar media, SB166, for 1-2 weeks. Clusters are then subcultured to medium SB103 for 3 weeks. During this period, individual embryos can be removed from the clusters and screened for levels of Zm-EREF-Ts expression and/or activity.

### Embryo Desiccation and Germination

Matured individual embryos are desiccated by placing them into an empty, small petri dish (35×10 mm) for approximately 4-7 days. The plates are sealed with fiber tape (creating a small humidity chamber). Desiccated embryos are planted into SB71-4 medium where they were left to germinate under the same culture conditions described above. Germinated plantlets are removed from germination medium and rinsed thoroughly with water and then planted in Redi-Earth in 24-cell pack tray, covered with clear plastic dome. After 2 weeks the dome is removed and plants hardened off for a further week. If plantlets looked hardy they are transplanted to 10″ pot of Redi-Earth with up to 3 plantlets per pot. After 10 to 16 weeks, mature seeds are harvested, chipped and analyzed for proteins.

### Media Recipes

SB 196-FN Lite liquid proliferation medium (per liter)-

| | | |
|---|---|---|
| MS FeEDTA-100x Stock 1 | 10 | ml |
| MS Sulfate-100x Stock 2 | 10 | ml |
| FN Lite Halides-100x Stock 3 | 10 | ml |
| FN Lite P, B, Mo-100x Stock 4 | 10 | ml |
| B5 vitamins (1 ml/L) | 1.0 | ml |
| 2,4-D (10 mg/L final concentration) | 1.0 | ml |
| KNO₃ | 2.83 | gm |
| (NH₄)₂SO₄ | 0.463 | gm |
| Asparagine | 1.0 | gm |
| Sucrose (1%) | 10 | gm |

pH 5.8

### FN Lite Stock Solutions

| Stock # | | 1000 ml | | 500 ml | |
|---|---|---|---|---|---|
| 1 | MS Fe EDTA 100x Stock | | | | |
| | Na₂ EDTA* | 3.724 | g | 1.862 | g |
| | FeSO₄—7H₂O | 2.784 | g | 1.392 | g |
| 2 | MS Sulfate 100x stock | | | | |
| | MgSO₄—7H₂O | 37.0 | g | 18.5 | g |
| | MnSO₄—H₂O | 1.69 | g | 0.845 | g |
| | ZnSO₄—7H₂O | 0.86 | g | 0.43 | g |
| | CuSO₄—5H₂O | 0.0025 | g | 0.00125 | g |
| 3 | FN Lite Halides 100x Stock | | | | |
| | CaCl₂—2H₂O | 30.0 | g | 15.0 | g |
| | KI | 0.083 | g | 0.0715 | g |
| | CoCl₂—6H₂O | 0.0025 | g | 0.00125 | g |
| 4 | FN Lite P, B, Mo 100x Stock | | | | |
| | KH₂PO₄ | 18.5 | g | 9.25 | g |
| | H₃BO₃ | 0.62 | g | 0.31 | g |
| | Na₂MoO₄—2H₂O | 0.025 | g | 0.0125 | g |

*Add first, dissolve in dark bottle while stirring

SB1 solid medium (per liter) comprises: 1 pkg. MS salts (GIBCO/BRL—Cat# 11117-066); 1 ml B5 vitamins 1000× stock; 31.5 g sucrose; 2 ml 2,4-D (20 mg/L final concentration); pH 5.7 and 8 g TC agar.

SB 166 solid medium (per liter) comprises: 1 pkg. MS salts (GIBCO/BRL—Cat# 11117-066); 1 ml B5 vitamins 1000× stock; 60 g maltose; 750 mg MgCl2 hexahydrate; 5 g activated charcoal; pH 5.7 and 2 g Gelrite®.

SB 103 solid medium (per liter) comprises: 1 pkg. MS salts (GIBCO/BRL—Cat# 11117-066); 1 ml B5 vitamins 1000× stock; 60 g maltose; 750 mg MgCl2 hexahydrate; pH 5.7 and 2 g Gelrite®.

SB 71-4 solid medium (per liter) comprises: 1 bottle Gamborg's B5 salts w/sucrose (GIBCO/BRL—Cat# 21153-036); pH 5.7 and 5 g TC agar.

2,4-D stock is obtained premade from Phytotech cat# D 295—concentration is 1 mg/ml.

B5 Vitamins Stock (per 100 ml) which is stored in aliquots at −20° C. comprises: 10 g myo-inositol; 100 mg nicotinic acid; 100 mg pyridoxine HCl and 1 g thiamine. If the solution does not dissolve quickly enough, apply a low level of heat via the hot stir plate.

Chlorsulfuron Stock comprises: 1 mg/ml in 0.01 N Ammonium Hydroxide.

### Example 5

### Rice Callus Transformation

Zm-EREF-Ts homologues from other crop species are analyzed by obtaining full-gene sequences. One method for transforming DNA into cells of higher plants that is available to those skilled in the art is high-velocity ballistic bombardment using metal particles coated with the nucleic acid constructs of interest (see, Klein, et al., (1987) Nature (London) 327:70-73 and see, U.S. Pat. No. 4,945,050). A Biolistic PDS-1000/He (BioRAD Laboratories, Hercules, Calif.) is used for these complementation experiments. The particle bombardment technique is used to transform the Zm-EREF-Ts mutants and wild type rice with two genomic DNA fragments:
1) 10.0 kb MunI fragment from wild type that includes the 4.5 kb upstream and 3.8 kb downstream region of the Zm-EREF-Ts gene,
2) 5.1 kb EcoRI fragment from wild type that includes the 1.7 kb upstream and 1.7 kb downstream region of the Zm-EREF-Ts gene.

The bacterial hygromycin B phosphotransferase (Hpt II) gene from Streptomyces hygroscopicus that confers resistance to the antibiotic is used as the selectable marker for rice transformation. In the vector, pML18, the Hpt II gene was engineered with the 35S promoter from Cauliflower Mosaic Virus and the termination and polyadenylation signals from the octopine synthase gene of Agrobacterium tumefaciens. pML18 was described in WO 97/47731, which was published on Dec. 18, 1997, the disclosure of which is hereby incorporated by reference.

Embryogenic callus cultures derived from the scutellum of germinating rice seeds serve as source material for transformation experiments. This material is generated by germinating sterile rice seeds on a callus initiation media (MS salts, Nitsch and Nitsch vitamins, 1.0 mg/l 2,4-D and 10 μM AgNO₃) in the dark at 27-28° C. Embryogenic callus proliferating from the scutellum of the embryos is the transferred to CM media (N6 salts, Nitsch and Nitsch vitamins, 1 mg/l 2,4-D, Chu, et al., (1985) Sci. Sinica 18:659-668). Callus cultures are maintained on CM by routine sub-culture at two week intervals and used for transformation within 10 weeks of initiation.

Callus is prepared for transformation by subculturing 0.5-1.0 mm pieces approximately 1 mm apart, arranged in a circular area of about 4 cm in diameter, in the center of a circle of Whatman® #541 paper placed on CM media. The plates with callus are incubated in the dark at 27-28° C. for 3-5 days. Prior to bombardment, the filters with callus are transferred to CM supplemented with 0.25 M mannitol and 0.25 M sorbitol for 3 hr in the dark. The petri dish lids are then left ajar for 20-45 minutes in a sterile hood to allow moisture on tissue to dissipate.

Each genomic DNA fragment is co-precipitated with pML18 containing the selectable marker for rice transformation onto the surface of gold particles. To accomplish this, a total of 10 μg of DNA at a 2:1 ratio of trait:selectable marker DNAs are added to 50 μl aliquot of gold particles that have been resuspended at a concentration of 60 mg ml⁻¹. Calcium chloride (50 μl of a 2.5 M solution) and spermidine (20 μl of a 0.1 M solution) are then added to the gold-DNA suspension as the tube is vortexing for 3 min. The gold particles are centrifuged in a microfuge for 1 sec and the supernatant removed. The gold particles are then washed twice with 1 ml of absolute ethanol and then resuspended in 50 μl of absolute ethanol and sonicated (bath sonicator) for one second to disperse the gold particles. The gold suspension is incubated at −70° C. for five minutes and sonicated (bath sonicator) if needed to disperse the particles. Six μl of the DNA-coated gold particles are then loaded onto mylar macrocarrier disks and the ethanol is allowed to evaporate.

At the end of the drying period, a petri dish containing the tissue is placed in the chamber of the PDS-1000/He. The air in the chamber is then evacuated to a vacuum of 28-29 inches Hg. The macrocarrier is accelerated with a helium shock wave using a rupture membrane that bursts when the He pressure in the shock tube reaches 1080-1100 psi. The tissue is placed approximately 8 cm from the stopping screen and the callus is bombarded two times. Two to four plates of tissue are bombarded in this way with the DNA-coated gold particles. Following bombardment, the callus tissue is transferred to CM media without supplemental sorbitol or mannitol.

Within 3-5 days after bombardment the callus tissue is transferred to SM media (CM medium containing 50 mg/l hygromycin). To accomplish this, callus tissue is transferred from plates to sterile 50 ml conical tubes and weighed. Molten top-agar at 40° C. is added using 2.5 ml of top agar/100 mg of callus. Callus clumps are broken into fragments of less than 2 mm diameter by repeated dispensing through a 10 ml pipet. Three ml aliquots of the callus suspension are plated onto fresh SM media and the plates are incubated in the dark for 4 weeks at 27-28° C. After 4 weeks, transgenic callus events are identified, transferred to fresh SM plates and grown for an additional 2 weeks in the dark at 27-28° C.

Growing callus is transferred to RM1 media (MS salts, Nitsch and Nitsch vitamins, 2% sucrose, 3% sorbitol, 0.4% Gelrite®+50 ppm hyg B) for 2 weeks in the dark at 25° C. After 2 weeks the callus is transferred to RM2 media (MS salts, Nitsch and Nitsch vitamins, 3% sucrose, 0.4% Gelrite®+50 ppm hyg B) and placed under cool white light (˜40 μEm⁻²s⁻¹) with a 12 hr photoperiod at 25° C. and 30-40% humidity. After 2-4 weeks in the light, callus begin to organize and form shoots. Shoots are removed from surrounding callus/media and gently transferred to RM3 media (½×MS salts, Nitsch and Nitsch vitamins, 1% sucrose+50 ppm hygromycin B) in Phytatrays™ (Sigma Chemical Co., St. Louis, Mo.) and incubation is continue using the same conditions as described in the previous step.

Plants are transferred from RM3 to 4″ pots containing Metro Mix® 350 after 2-3 weeks, when sufficient root and shoot growth have occurred. The seed obtained from the transgenic plants is examined for genetic complementation of the Zm-EREF-Ts mutation with the wild-type genomic DNA containing the Zm-EREF-Ts gene.

### Example 6

### Over expression of Zm-EREF-Ts Increased Kernel Number and Total Kernel Weight Per Plant in Corn

Data collected from T0 corn transgenic plants for Ubi-Zm-EREF-Ts showed that the overexpression of Zm-EREF-Ts in maize could enhance plant growth rate, kernel number per ear and total kernel weight per plant. 9 out of 10 transgenic plants containing ubi-Zm-EREF-Ts have more kernel numbers per ear and have total kernel weight higher than the average of all transgenic events evaluated. 10 out of 10 transgenic plants containing ubi-Zm-EREF-Ts have higher plant growth rate than the average of all transgenic events evaluated.

Transgene expression analysis was carried out for all plants from 3 events evaluated in T1 yield assay where 13 transgene positive plants from each event are evaluated in comparison with a reference population composed of non-transgenic segregants from the same events. The transgenic plants have significantly more silk and significantly higher specific growth rate than their non-transgenic segregants (see Table 2) in two events where the transgene are expressed. No significantly increases in the same parameters were observed in the event where transgene was not expressed. Ubi-Zm-EREF-Ts transformed into elite corn showed positive yield increases in some locations but none of the increases were significant.

TABLE 2

| | | % | | increase in | | |
|---|---|---|---|---|---|---|
| | | increase | | specific | | Relative |
| Con- | | in silk | signifi- | growth | signifi- | gene |
| struct | Event | number | cance | rate | cance | expression |
| Ubi- | 1 | 4.03% | NS | 3.79% | NS | −0.13 |
| ZmEREF- | 2 | 24.51% | P < 0.05 | 6.85% | P < 0.05 | 5.00 |
| Ts | 3 | 25.75% | P < 0.05 | 18.58% | P < 0.05 | 6.28 |

### Example 7

### Overexpression of Zm-EREF-Ts Increased Growth Rate and Seed Yield in Soybean

Data collected from soybean transgenic plants over-expressing AT-UBQ10::Zm-EREF-Ts showed that Zm-EREF-Ts could enhance plant growth rate, plant biomass and total seed number in soybean. Quantitative RT-PCR was performed with T0 and T1 plants and transgene expression was confirmed in all events. Five events are evaluated in T1 generation where 10 transgene positive plants from each event are evaluated in comparison with a reference population composed of non-transgenic segregants from the same event.

Near-isogenic lines (isolines) of AT-UBQ10::Zm-EREF-Ts events are created by selfing the T0 and T1 generation and identifying T2 plants that are either homozygous positive for presence of transgene or homozygous negative (lacking the transgene). Zygosity of each T2 plant is determined by using quantitative PCR to determine copy number of the transgene. Seed of selected T2 plants is increased as single plant-to-row in the T3 generation to generate seed for preliminary yield testing in year 1. Seed of each isoline from the T4 and T5 generation is increased for advanced level yield testing in year 2 and 3, respectively. Statistical analyses for yield data is completed using the mixed model of SAS® (SAS Institute, Inc., Cary, N.C., USA). When yield data is examined, positive isolines have a significantly higher yield compared to negative isolines in the field.

### Example 8

### Variants of Zm-EREF-Ts Sequences

A. Variant Nucleotide Sequences of Zm-EREF-Ts that do not Alter the Encoded Amino Acid Sequence

The Zm-EREF-Ts nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO: 1. These functional variants are generated using a standard codon table. While the nucleotide sequence of the variants are altered, the amino acid sequence encoded by the open reading frames do not change.

B. Variant Amino Acid Sequences of Zm-EREF-Ts Polypeptides

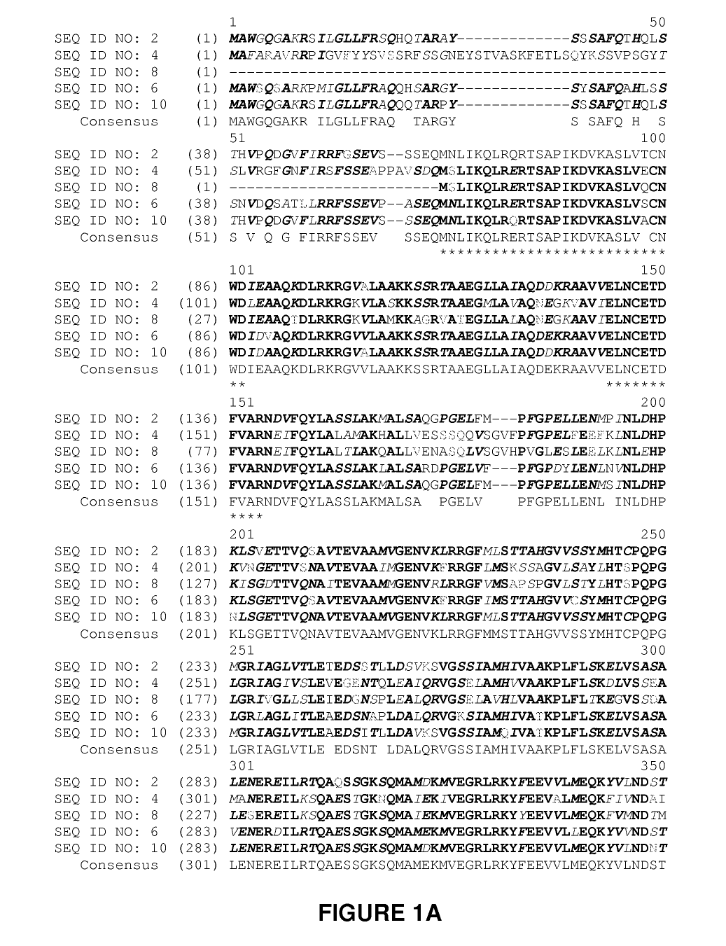

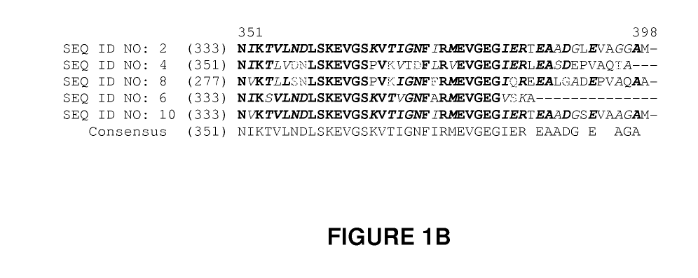

Variant amino acid sequences of the Zm-EREF-Ts polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using the protein alignment set forth in FIG. 1, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

C. Additional Variant Amino Acid Sequences of Zm-EREF-Ts Polypeptides

In this example, artificial protein sequences are created having 80%, 85%, 90% and 95% identity relative to the reference protein sequence. This latter effort requires identifying conserved and variable regions from the alignment set forth in FIG. 1 and then the judicious application of an amino acid substitutions table. These parts will be discussed in more detail below.

Largely, the determination of which amino acid sequences are altered is made based on the conserved regions among Zm-EREF-Ts protein or among the other Zm-EREF-Ts polypeptides. Based on the sequence alignment, the various regions of the Zm-EREF-Ts polypeptide that can likely be altered are represented in lower case letters, while the conserved regions are represented by capital letters. It is recognized that conservative substitutions can be made in the conserved regions below without altering function. In addition, one of skill will understand that functional variants of the Zm-EREF-Ts sequence of the invention can have minor non-conserved amino acid alterations in the conserved.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 3.

TABLE 3
Substitution Table

| | Strongly Similar | Rank of | |
|---|---|---|---|
| Amino | and Optimal | Order to | |
| Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the Zm-EREF-Ts polypeptides are generating having about 80%, 85%, 90% and 95% amino acid identity to the starting unaltered ORF nucleotide sequence of SEQ ID NO: 1.

D. Disruption of Targeted Domains or Sequences of Zm-EREF-Ts Polypeptides

Disrupted amino acid sequences of the Zm-EREF-Ts polypeptides are generated. In this example, particular domains are disrupted or excluded from final polypeptide. If disrupting the N-terminal domain(s) or motif(s), the DNA codon for the starting ATG is altered by insertion, deletion or base substitution to prevent the translation of the first methionine. Generally the next available methionine will dominate the start of translation thus skipping the N-terminal portion of the polypeptide. For Zm-EREF-Ts gene, the first two ATG can be altered to effectively prevent translation starting at these ATG and initiating downstream at nucleotide position 530 of SEQ ID NO: 1 thus eliminating the translation of the first 152 amino acids of SEQ ID NO: 2. If disrupting a C-terminal domain, a stop codon at the desired site is created by insertion, deletion or base substitution or more commonly by PCR as described below. Premature stops may lead to translation of polypeptides missing the C-terminal domain(s).

An alternative method for selectively isolating a targeted region(s) for expression is to design primers to PCR amplify the desired domain(s) with either a naturally occurring or engineered ATG sequence at the 5′ end of the clone and a naturally occurring or engineered stop codon at the 3′ end of the clone. The resulting fragment will have the desired domain(s) to be cloned into expression vectors. For SEQ ID NO: 1, a PCR 5′ primer was designed to be complementary to the region starting at position 251 and contained an in-frame ATG codon while the PCR 3′ primer was designed to be complementary to the region ending at position 532 with the stop codon. The resulting PCR fragment will have the desired region(s) to be cloned into expression vectors.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

That which is claimed:

1. An isolated polynucleotide comprising a nucleotide sequence selected from the group consisting of:
(a) the nucleotide sequence set forth in SEQ ID NO: 1;
(b) a nucleotide sequence encoding the amino acid sequence of SEQ ID NO: 2;
(c) a nucleotide sequence having at least 90% sequence identity to SEQ ID NO: 1, wherein said nucleotide sequence encodes a polypeptide having Zm-EREF-Ts protein activity;
(d) a nucleotide sequence comprising at least 99 consecutive nucleotides of SEQ ID NO: 1 or a complement thereof; and
(e) a nucleotide sequence encoding an amino acid sequence having at least 80% sequence identity to SEQ ID NO: 2, wherein said nucleotide sequence encodes a polypeptide having Zm-EREF-Ts protein activity.

2. An expression cassette comprising the polynucleotide of claim 1.

3. The expression cassette of claim 2, wherein said polynucleotide is operably linked to a promoter that drives expression in a plant.

4. The expression cassette of claim 3, wherein said polynucleotide is operably linked to a constitutive promoter.

5. A plant comprising the expression cassette of claim 3 or claim 4.

6. The plant of claim 5, wherein said plant is a monocot.

7. The plant of claim 6, wherein said monocot is maize, wheat, rice, barley, sorghum or rye.

8. The plant of claim 7, wherein said monocot is rice.

9. The plant of claim 7, wherein said monocot is maize.

10. The plant of claim 5, wherein said plant has an increased level of a polypeptide selected from the group consisting of:
(a) a polypeptide comprising the amino acid sequence of SEQ ID NO: 2;
(b) a polypeptide having at least 90% sequence identity to SEQ ID NO: 2, wherein said polypeptide has Zm-EREF-Ts protein activity; and
(c) a polypeptide comprising a 28 amino acid sequence having at least 90% sequence identity to the Zm-EREF-Ts conserved consensus sequence set forth in SEQ ID NO: 12 and a elongation factor Ts signature set forth in SEQ ID NO: 13.

11. The plant of claim 5, wherein said plant has a phenotype selected from the group consisting of:
(a) an increased in plant growth rate;
(b) an increase in total seed number; and
(b) an increased total seed weight;

12. A method of increasing the level of a polypeptide in a plant comprising introducing into said plant the expression cassette of claim 3 or claim 4.

13. The method of claim 12, wherein the yield of the plant is increased.

14. The method of claim 12, wherein increasing the level of said polypeptide produces a phenotype in the plant selected from the group consisting of:
(a) an increased in plant growth rate;
(b) an increase in total seed number; and
(c) an increased total seed weight;

15. The method of claim 13, wherein said expression cassette is stably integrated into the genome of the plant.

16. The method of claim 13, wherein said plant is a monocot.

17. The method of claim 16, wherein said monocot is maize, wheat, rice, barley, sorghum or rye.

18. The method of claim 17, wherein said monocot is rice.

19. The method of claim 17, wherein said monocot is maize.

20. A method of increasing yield in a plant comprising increasing expression of a Zm-EREF-Ts polypeptide in said plant, wherein said Zm-EREF-Ts polypeptide has Zm-EREF-Ts protein activity and is selected from the group consisting of:
(a) a polypeptide comprising an amino acid sequence having at least 80% sequence identity to the sequence set forth in SEQ ID NO: 2; and
(b) a polypeptide comprising a 28 amino acid sequence having at least 90% sequence identity to the Zm-EREF-Ts conserved consensus sequence set forth in SEQ ID NO: 12 and a elongation factor Ts signature set forth in SEQ ID NO: 13.

21. The method of claim 20, wherein said polypeptide comprises an amino acid sequence having at least 95% sequence identity with the sequence set forth in SEQ ID NO: 2.

22. The method of claim 20, wherein said polypeptide comprises the amino acid sequence set forth in SEQ ID NO: 2.

23. The method of claim 20, comprising introducing into said plant an expression cassette comprising a polynucleotide encoding said Zm-EREF-Ts polypeptide operably linked to a promoter that drives expression in a plant cell, wherein said polynucleotide comprises a nucleotide sequence selected from the group consisting of:
(a) the nucleotide sequence set forth in SEQ ID NO: 1;
(b) a nucleotide sequence encoding the polypeptide of SEQ ID NO: 2;
(c) a nucleotide sequence comprising at least 95% sequence identity to the sequence set forth in SEQ ID NO: 1;
(d) a polypeptide comprising a 28 amino acid sequence having at least 90% sequence identity to the Zm-EREF-Ts conserved consensus sequence set forth in SEQ ID NO: 12 and a elongation factor Ts signature set forth in SEQ ID NO: 13;
(e) a nucleotide sequence encoding an amino acid sequence having at least 90% sequence identity to the sequence set forth in SEQ ID NO: 2.

24. The method of claim 23, comprising:
(a) transforming a plant cell with said expression cassette; and
(b) regenerating a transformed plant from the transformed plant cell of step (a).

25. The method of claim 23, wherein said expression cassette is stably incorporated into the sequence of the plant.

26. The method of claim 23, wherein said promoter is a constitutive promoter.

27. An isolated polypeptide comprising an amino acid sequence selected from the group consisting of:
(a) the amino acid sequence comprising SEQ ID NO: 2;
(b) the amino acid sequence comprising at least 90% sequence identity to SEQ ID NO: 2, wherein said polypeptide has the ability to modulate transcription; and
(c) the amino acid sequence comprising at least 40 consecutive amino acids of SEQ ID NO: 2, wherein said polypeptide retains the ability to modulate transcription.


SEQUENCE LISTING (13 sequences)

SEQ ID NO: 1 - DNA, 1524 bp, Zea mays
agatgcccagttcttcgtcagccccttctccggcaacgagcacctaacagaggctgcagtatattctgtgagaatggcatggggtcaaggtgctaagaggtccatactagggcttctctttcgttctcagcaccaaactgctcgggcatactcttcttcagcattccagactcaccaattgagcactcatgttccccaagatggagtgtttattaggagattcggctctgaggtgtcttcttcagagcaaatgaaccttattaagcaactcagacaaagaacgagtgctccaatcaaagatgtcaaggcctctctagttacctgcaactgggatattgaggctgcacagaaggacctaaggaagagaggtgttgctcttgctgccaaaaagtcttcacgcactgctgctgaaggtttgctagctatcgcacaagatgacaaaagggctgctgtagttgagcttaactgtgaaactgatttcgtagcaagaaatgatgttttccagtacctggcttcatcattggcaaagatggctttatctgctcagggtcctggtgaattgttcatgccttttggtcctgagcttttagagaacatgcctatcaatctagatcaccctaagctcagtgtggaaacaactgtccaaagtgccgttacagaagttgctgcaatggttggggagaatgtgaaactcagaagaggcttcatgctttccacaactgcacatggtgttgtttcatcttatatgcatacctgccctcagccaggtatgggtcgtatagctggattggtcacactagaaacagaagacagcagtactcttcttgattctgtcaaaagtgttgggtcatctattgcgatgcatattgttgcggcaaagccattattcttatcaaaagaactggtttctgcttcggctttagaaaatgagcgtgaaatacttcgaactcaggcccaaagttcagggaaatcccaaatggctatggataagatggtggaaggacgattgaggaagtactttgaggaagttgtgctcatggagcaaaaatatgttttaaatgacagcactaacattaagaccgtgctgaatgacttgtcgaaagaggttggttctaaggtaacaatcggtaacttcatcagaatggaggttggcgaagggattgagagaactgaagccgctgatggtttagaggttgctggtggcgctatgtagattccaggcagagatggttcgaacgagatcattaggatctatatgaaggtcccacttctctaaagttggggggccttggttttttccaataaaacttttgggctctcatctttgctccattggattgttacaggtgtgggctaggacggagataatgtttaggcaggcactagcgttctgggacctgcgaggctgcctagttgatttgtcagttcctagcttgtttggttacagttttggttgaaccctctctgattgaattacagttcgtgtggacgtataaaaaaaaaaaaaaaaaaaaaaaaaaaaaa

SEQ ID NO: 2 - protein, 379 aa, Zea mays
MAWGQGAKRSILGLLFRSQHQTARAYSSSAFQTHQLSTHVPQDGVFIRRFGSEVSSSEQMNLIKQLRQRTSAPIKDVKASLVTCNWDIEAAQKDLRKRGVALAAKKSSRTAAEGLLAIAQDDKRAAVVELNCETDFVARNDVFQYLASSLAKMALSAQGPGELFMPFGPELLENMPINLDHPKLSVETTVQSAVTEVAAMVGENVKLRRGFMLSTTAHGVVSSYMHTCPQPGMGRIAGLVTLETEDSSTLLDSVKSVGSSIAMHIVAAKPLFLSKELVSASALENEREILRTQAQSSGKSQMAMDKMVEGRLRKYFEEVVLMEQKYVLNDSTNIKTVLNDLSKEVGSKVTIGNFIRMEVGEGIERTEAADGLEVAGGAM

SEQ ID NO: 3 - DNA, 1492 bp, Arabidopsis thaliana
aaaaccatcaccgtcgttatcacagtaaccaaacaactttcatcgccggagaactgattaacatataagaaaatggcgtttgctagagcagtgagaagaccaattggagtcttttattatagtgttagtagtagatttagttctggaaatgagtattctactgtggcaagcaaattcgaaactttgtctcagtacaaaagctctgtaccgtctggttacacaagccttgttcgtggttttgggaatttcatacggagttttagttcggaagctcctcctgctgtatctgaccaaatgagtcttattaagcaacttagggaaagaactagtgctcctattaaagatgttaaggcttctttagttgaatgcaattgggatcttgaggctgctcagaaggatttgagaaagagagggaaagttttggcttcgaaaaagtcgtcacggactgctgcagaaggaatgcttgccgttgctcaaaatgaagggaaagttgctgttattgaacttaactgtgagactgattttgttgctaggaatgagattttccagtacttggctttagctatggcaaaacatgctttgctggttgagagtagttcgcagcaagtttctggtgtcttcccctttgggccggagctttttgaggagttcaagcttaacctcgatcatccaaaagttaatggtgaaacaacggtttcaaacgctgttacagaagtagctgcaatcatgggagagaatgtaaaatttaggagaggtttcttgatgtcaaaatcttcagcaggcgtcctttctgcatatcttcacacaagtcctcaaccagggttgggtcgtatagctgggattgtatctctcgaagtagaaggtgaaaatactcaactagaggctattcagcgtgtaggctcagaactggctatgcatgttgtagcagcaaagcctttgtttttaagcaaagaccttgtttcttctgaagcaatggcaaatgaacgcgagattctaaagtctcaggcagaaagtacaggcaagaatcagatggcgatagagaagattgtggaagggcgtctccgtaagtattttgaagaagttgctctaatggagcaaaagtttattgtgaacgatgctataaacattaagacactggtggataatttgtccaaggaggtaggttctccagtgaaggttaccgattttcttagagtcgaggttggggaaggaatcgaaaggcttgaagcatctgatgaaccagttgctcaaactgcttgaagacaaagaatcaacgacatggttttagatttacagaactctttccttctttggttcctttagagttcatggcttcaacgccttcttgagattatagaatctttctcatgattttgtttctcacacaatatggcaataagagtgattgtgcctaggaagctatgaccagaaaaaaaaaacatgtttgtgttctcctactcctactaagatcattatacaaaaaccttcactt

SEQ ID NO: 4 - protein, 395 aa, Arabidopsis thaliana
MAFARAVRRPIGVFYYSVSSRFSSGNEYSTVASKFETLSQYKSSVPSGYTSLVRGFGNFIRSFSSEAPPAVSDQMSLIKQLRERTSAPIKDVKASLVECNWDLEAAQKDLRKRGKVLASKKSSRTAAEGMLAVAQNEGKVAVIELNCETDFVARNEIFQYLALAMAKHALLVESSSQQVSGVFPFGPELFEEFKLNLDHPKVNGETTVSNAVTEVAAIMGENVKFRRGFLMSKSSAGVLSAYLHTSPQPGLGRIAGIVSLEVEGENTQLEAIQRVGSELAMHVVAAKPLFLSKDLVSSEAMANEREILKSQAESTGKNQMAIEKIVEGRLRKYFEEVALMEQKFIVNDAINIKTLVDNLSKEVGSPVKVTDFLRVEVGEGIERLEASDEPVAQTA

SEQ ID NO: 5 - DNA, 1101 bp, Oryza sativa
atggcctggagtcagagtgctagaaagcccatgattgggcttctgtttcgcgcccaacaacattctgctcggggttactcttattcagcgttccaggctcacctttcgagcagcaatgtcgatcaaagcgccacacttctcaggagattcagctccgaagttcctgcgtcggagcaaatgaatctcatcaaacagctgagggagaggacgagcgctccaatcaaagatgtgaaggcttccttagttagttgcaactgggatattgatgttgcacagaaggacctaaggaagagaggggtggttcttgctgccaaaaagtcttcacggactgcagctgaaggtttgcttgctattgcacaagatgagaaaagggccgctgtggttgagcttaactgtgagacggattttgtggcaagaaatgatgttttccagtatctggcttcttctttagcaaagttggctttatcagcaagggatcctggtgaattggtttttccttttggtcctgactatttggagaaccttaatgtcaatcttgatcatcctaaacttagtggggagacaacagtccaaagtgctgtcacagaagttgctgccatggttggggagaatgtcaaattcagaagaggtttcattatgtccacaactgcacatggtgttgtttgctcttatatgcacacatgtccccagccaggtttgggtcgtcttgctggcttgatcacactagaggcagaagacagcaatgctcctcttgatgctcttcagagagtcgggaaatctattgcaatgcacatcgttgcgacaaagccgttgtttttatcaaaagaactggtctctgcttcagctgttgaaaatgagagggacatactccggactcaggctgaaagctctggaaaatcccaaatggccatggagaaaatggtggaaggccgactaagaaagtattttgaagaagttgtgcttttggagcaaaaatatgttgtcaatgacagcacaaacatcaagagcgtcctgaatgatttgtcaaaggaagttggctcaaaagttacagttggcaattttgctaggatggaagttggcgaaggagtttcaaaggcctga

SEQ ID NO: 6 - protein, 366 aa, Oryza sativa
MAWSQSARKPMIGLLFRAQQHSARGYSYSAFQAHLSSSNVDQSATLLRRFSSEVPASEQMNLIKQLRERTSAPIKDVKASLVSCNWDIDVAQKDLRKRGVVLAAKKSSRTAAEGLLAIAQDEKRAAVVELNCETDFVARNDVFQYLASSLAKLALSARDPGELVFPFGPDYLENLNVNLDHPKLSGETTVQSAVTEVAAMVGENVKFRRGFIMSTTAHGVVCSYMHTCPQPGLGRLAGLITLEAEDSNAPLDALQRVGKSIAMHIVATKPLFLSKELVSASAVENERDILRTQAESSGKSQMAMEKMVEGRLRKYFEEVVLLEQKYVVNDSTNIKSVLNDLSKEVGSKVTVGNFARMEVGEGVSKA

SEQ ID NO: 7 - DNA, 972 bp, Populus truncatula
atgagtctcattaagcaattgagagaacggactagcgctcctatcaaagatgtcaaggcttctcttgtccaatgcaattgggatattgaggcggcacagacggatttgaggaaaagagggaaagttttggcaatgaaaaaggcgggtagagttgccactgaaggattgcttgcattggctcagaatgaggggaaggctgctgttattgaacttaattgtgaaactgactttgttgccagaaatgaaattttccaatacttggctttaactttagcaaagcaggctttgctggttgaaaatgcttctcagctggtttcgggggtccaccctgtaggtctcgagtctctggaggagttgaagttaaatcttgaacatccaaaaataagtggagatacaactgttcaaaatgcaattacagaagtggctgcaatgatgggtgagaatgtaagactcagaaggggctttgtgatgtctgcaccttcacccggtgttctttccacttaccttcatactagtcctcaaccaggtttgggccgtattgtgggacttttatctcttgagatagaagatggaaattctccgttggaggctctccagcgtgttggatcagaattggctgtgcatctggtagctgcaaaaccattattcttgactaaggaaggtgtttcctctgatgcattagagagtgaacgtgaaattcttaagtctcaggcagaaagtacagggaagtctcaaatggccatagagaaaatggtggaaggccgtttgcgtaaatactacgaggaagttgttttgatggagcaaaaatttgttatgaatgatacaatgaacgtaaagacactgctcagcaatctatcgaaggaagtgggctcgcctgtgaagataggtaactttttcagaatggaagttggagagggaattcaaagggaggaggcgttgggtgcagatgaaccagtggctcaggcagcttga

SEQ ID NO: 8 - protein, 323 aa, Populus truncatula
MSLIKQLRERTSAPIKDVKASLVQCNWDIEAAQTDLRKRGKVLAMKKAGRVATEGLLALAQNEGKAAVIELNCETDFVARNEIFQYLALTLAKQALLVENASQLVSGVHPVGLESLEELKLNLEHPKISGDTTVQNAITEVAAMMGENVRLRRGFVMSAPSPGVLSTYLHTSPQPGLGRIVGLLSLEIEDGNSPLEALQRVGSELAVHLVAAKPLFLTKEGVSSDALESEREILKSQAESTGKSQMAIEKMVEGRLRKYYEEVVLMEQKFVMNDTMNVKTLLSNLSKEVGSPVKIGNFFRMEVGEGIQREEALGADEPVAQAA

SEQ ID NO: 9 - DNA, 1140 bp, Sorghum bicolor
atggcctggggtcaaggtgctaagaggtccatactagggcttctctttcgtgctcagcagcaaactgctcggccatactcttcttcagcattccagactcatcaattgagcactcatgttccccaagatggcgtgtttcttaggagattcagctctgaggtgtcttcttcagagcaaatgaaccttataaagcaactcagacaaaggacgagtgctccaatcaaagatgtcaaggcctccctagttgcctgcaactgggatattgatgctgcgcagaaggacctaaggaagagaggtgttgctcttgctgcaaaaaagtcttcacgcactgctgctgaaggtttgctagctatcgcacaagatgacaaaagggctgctgtagttgagcttaactgtgaaactgatttcgtggcaagaaatgatgttttccagtacctggcttcatcattggcaaagatggctttatctgctcagggtcccggtgaattgttcatgccttttggtcctgaacttttagagaacatgtctatcaatcttgatcatccgaacctcagcggggaaacaactgtccaaaatgctgttacagaagttgctgccatggttggggagaatgtgaaactcagaagaggcttcatgctgtccacaactgcacatggtgttgtttcatcttatatgcatacctgtccccagccaggtatgggtcggattgctggattggtcacactagaagcagaagatagcattactctccttgatgctgtcaaaagtgttgggtcatctattgcgatgcagattgttgcaacaaagccattattcttatcaaaagaactggtttctgcttccgctctagaaaatgagcgtgaaatacttcgaacacaggccgaaagttcagggaaatcccaaatggctatggataaaatggtggaaggccgattgaggaagtactttgaagaagttgtgctcatggagcaaaaatatgttttaaatgacaacacaaacgtgaagaccgtgctgaatgacttgtcgaaagaggttggttctaaagtaacaatcggtaacttcatcagaatggaggttggagaagggattgagagaactgaagccgctgatggttcagaggttgctgctggggctatgtag

SEQ ID NO: 10 - protein, 379 aa, Sorghum bicolor
MAWGQGAKRSILGLLFRAQQQTARPYSSSAFQTHQLSTHVPQDGVFLRRFSSEVSSSEQMNLIKQLRQRTSAPIKDVKASLVACNWDIDAAQKDLRKRGVALAAKKSSRTAAEGLLAIAQDDKRAAVVELNCETDFVARNDVFQYLASSLAKMALSAQGPGELFMPFGPELLENMSINLDHPNLSGETTVQNAVTEVAAMVGENVKLRRGFMLSTTAHGVVSSYMHTCPQPGMGRIAGLVTLEAEDSITLLDAVKSVGSSIAMQIVATKPLFLSKELVSASALENEREILRTQAESSGKSQMAMDKMVEGRLRKYFEEVVLMEQKYVLNDNTNVKTVLNDLSKEVGSKVTIGNFIRMEVGEGIERTEAADGSEVAAGAM

SEQ ID NO: 11 - DNA, 1140 bp, Zea mays
atggcatggggtcaaggtgctaagaggtccatactagggcttctctttcgttctcagcaccaaactgctcgggcatactcttcttcagcattccagactcaccaattgagcactcatgttccccaagatggagtgtttattaggagattcggctctgaggtgtcttcttcagagcaaatgaaccttattaagcaactcagacaaagaacgagtgctccaatcaaagatgtcaaggcctctctagttacctgcaactgggatattgaggctgcacagaaggacctaaggaagagaggtgttgctcttgctgccaaaaagtcttcacgcactgctgctgaaggtttgctagctatcgcacaagatgacaaaagggctgctgtagttgagcttaactgtgaaactgatttcgtagcaagaaatgatgttttccagtacctggcttcatcattggcaaagatggctttatctgctcagggtcctggtgaattgttcatgccttttggtcctgagcttttagagaacatgcctatcaatctagatcaccctaagctcagtgtggaaacaactgtccaaagtgccgttacagaagttgctgcaatggttggggagaatgtgaaactcagaagaggcttcatgctttccacaactgcacatggtgttgtttcatcttatatgcatacctgccctcagccaggtatgggtcgtatagctggattggtcacactagaaacagaagacagcagtactcttcttgattctgtcaaaagtgttgggtcatctattgcgatgcatattgttgcggcaaagccattattcttatcaaaagaactggtttctgcttcggctttagaaaatgagcgtgaaatacttcgaactcaggcccaaagttcagggaaatcccaaatggctatggataagatggtggaaggacgattgaggaagtactttgaggaagttgtgctcatggagcaaaaatatgttttaaatgacagcactaacattaagaccgtgctgaatgacttgtcgaaagaggttggttctaaggtaacaatcggtaacttcatcagaatggaggttggcgaagggattgagagaactgaagccgctgatggtttagaggttgctggtggcgctatgtag

SEQ ID NO: 12 - protein, 28 aa, Artificial Sequence
  conserved sequence
MNLIKQLRERTSAPIKDVKASLVXCNWD

SEQ ID NO: 13 - protein, 11 aa, Artificial Sequence
  elongation factor Ts signature
ELNCETDFVAR